(19) United States
(12) Patent Application Publication
(10) Pub. No.: US 2025/0121332 A1
(43) Pub. Date: Apr. 17, 2025

(54) ARTICLES, SYSTEMS, AND METHODS RELATED TO NANOPOROUS MEMBRANES

(71) Applicant: Massachusetts Institute of Technology, Cambridge, MA (US)

(72) Inventors: Rohit N. Karnik, Cambridge, MA (US); Aaron Persad, Somerville, MA (US); Chun Man Chow, Cambridge, MA (US); Chi David Cheng, Providence, RI (US)

(73) Assignee: Massachusetts Institute of Technology, Cambridge, MA (US)

(21) Appl. No.: 18/688,053

(22) PCT Filed: Sep. 7, 2022
(86) PCT No.: PCT/US2022/042796

§ 371 Date: Feb. 29, 2024

Related U.S. Application Data
(60) Provisional application No. 63/241,922, filed on Sep. 8, 2021.

(51) Int. Cl. B01D 69/12; A61M 1/16; B01D 61/24; B01D 69/06; B01D 71/02; B01D 71/56; B01D 71/68; B82Y 5/00

(52) U.S. Cl. CPC B01D 69/1216; A61M 1/1631; B01D 61/243; B01D 69/06; B01D 71/0211; B01D 71/0213; B01D 71/56; B01D 71/68; A61M 2202/0445; A61M 2202/0496; B01D 2325/02832; B01D 2325/02833; B01D 2325/02834; B01D 2325/04; B82Y 5/00

(57) ABSTRACT

Articles, systems, and methods related to the separation of at least a first species from at least a second species using nanoporous membranes are generally described.

### CROSS-REFERENCE TO RELATED APPLICATIONS

This application claims the benefit under 35 U.S.C. § 119(e) of U.S. provisional application Ser. No. 63/241,922, filed Sep. 8, 2021, the disclosure of which is incorporated by reference in its entirety.

### TECHNICAL FIELD

Articles, systems, and methods related to the separation of at least a first species from at least a second species using nanoporous membranes are generally described.

### BACKGROUND

Many industries and applications, such as water purification, chemical synthesis, pharmaceutical purification, refining, and natural gas separation, utilize membrane-based separation processes. The need for membranes with high selectivity and flux for both liquid-phase and gas-phase membranes has led to many improvements in ceramic and polymer-based membranes over the past few decades. One of the primary challenges has been to maximize flux while maintaining high selectivity. Typically, increasing flux rate necessitates a decrease in selectivity. While several decades of research has resulted in development of polymeric or ceramic membranes, further advances in membrane technology will likely rely on new membrane materials that provide better transport properties. Recent advances in two-dimensional (2D) materials such as graphene have opened new opportunities to advance membrane technology, where these 2D materials can form the active layer of the membrane that confers selectivity.

### SUMMARY

Articles, systems, and methods related to the separation of at least a first species from at least a second species using nanoporous membranes are generally described. The subject matter of the present disclosure involves, in some cases, interrelated products, alternative solutions to a particular problem, and/or a plurality of different uses of one or more systems and/or articles.

According to some embodiments, a semi-permeable membrane is described, the semi-permeable membrane comprising an atomically thin layer, wherein the atomically thin layer comprises a plurality of pores that allow transport of at least a first species though the semi-permeable membrane while restricting transport of at least a second species through the semi-permeable membrane, a porous intermediate coating disposed on the atomically thin layer, and a porous substrate, wherein the porous intermediate coating is disposed between the atomically thin layer and the porous substrate.

In certain embodiments, a dialysis system is described, the dialysis system comprising a first compartment configured to receive a flow of blood, a second compartment configured to receive a flow of a dialysate, and a semi-permeable membrane disposed between the first compartment and the second compartment. In some embodiments, the semi-permeable membrane comprises an atomically thin layer, wherein the atomically thin layer comprises a plurality of pores that allow transport of at least a first species though the semi-permeable membrane while restricting transport of at least a second species through the semi-permeable membrane, a porous intermediate coating disposed on the atomically thin layer, and a porous substrate, wherein the porous intermediate coating is disposed between the atomically thing layer and the porous substrate. In some embodiments, the dialysis system is configured such that at least the first species is transported from the first compartment into the second compartment though the semi-permeable membrane.

According to some embodiments, a method of performing dialysis is described, the method comprising separating at least a first species from at least a second species using a semi-permeable membrane. In certain embodiments, the semi-permeable membrane comprises an atomically thin layer, wherein the atomically thin layer comprises a plurality of pores that allow transport of at least the first species though the semi-permeable membrane while restricting transport of at least the second species through the semi-permeable membrane, a porous intermediate coating disposed on the atomically thin layer, and a porous substrate, wherein the porous intermediate coating is disposed between the atomically thin layer and the porous substrate. In some embodiments, at least the first species passes through the semi-permeable membrane via diffusion.

Other advantages and novel features of the present disclosure will become apparent from the following detailed description of various non-limiting embodiments of the disclosure when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present disclosure will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIG. 1A shows, according to some embodiments, a schematic diagram of a semi-permeable membrane;

FIG. 1B shows, according to some embodiments, an expanded schematic diagram of the semi-permeable membrane shown in FIG. 1A;

FIGS. 2A-2E show, according to some embodiments, a method of fabricating a semi-permeable membrane;

FIG. 3 shows, according to some embodiments, a schematic diagram of a dialysis system;

FIG. 4 shows, according to some embodiments, a schematic diagram of a dialysis system wherein the dialysate comprises an adsorbent;

FIG. 5A shows, according to some embodiments, a top-view scanning electron microscopy (SEM) image of a porous coating;

FIG. 5B shows, according to some embodiments, a cross-sectional SEM image of a porous coating; and

FIG. 6 shows, according to some embodiments, a schematic diagram of a dialysis device;

FIG. 7 shows, according to some embodiments, a computer-aided design of a dialysis device;

FIG. 8 shows, according to some embodiments, a dialysis device;

FIG. 9 shows, according to some embodiments, the measurement of protein-bound uremic toxins transport through a composite membrane;

FIG. 10 shows, according to some embodiments, a schematic diagram of a diffusion experimental setup with a composite membrane; and

FIG. 11 shows, according to some embodiments, indoxyl sulfate (IS) and bovine serum albumin (BSA) permeance and corresponding IS/BSA selectivity for various oxygen plasma etch durations of a composite membrane compared to unmodified commercial polysulfone (PS) membranes;

FIG. 12A shows, according to some embodiments, a nylon mesh with its peripheral areas embedded in a polydimethylsiloxane layer that facilitates sealing around the periphery;

FIG. 12B shows, according to some embodiments, an optical microscope image of the nylon mesh in FIG. 12A; and

FIG. 13 shows, according to some embodiments, a composite membrane including graphene on polyethersulfone on nylon mesh (Nylon-PES-G) placed on a silicone gasket on a diffusion cell.

### DETAILED DESCRIPTION

Hemodialysis treatment plays a major role in reducing the mortality rate of patients with acute or chronic kidney failure. Conventional hemodialysis technology, however, achieves less than ideal clearance of certain target molecules, such as uremic toxins (e.g., p-cresyl sulfate, indoxyl sulfate), especially those bound to proteins (e.g., albumin) in blood. The Inventors have recognized that improved clearance of such uremic toxins has the potential to significantly reduce the long-term mortality rate of patients undergoing hemodialysis.

Conventional hemodialysis systems utilize filter cartridges consisting of, for example, symmetric membranes comprising modified cellulose or asymmetric membranes comprising synthetic hollow-fibers. The symmetric membranes have substantially homogenous pore sizes that extend through the thickness of the membrane, with thicker selective layers that result in high diffusive resistance. Asymmetric membranes contain thin inner selective layers and thick outer support layers, in which the porosity of the support layer may be increased, resulting in higher transport rates as compared to the symmetric membranes. Challenges associated with conventional membranes, however, include the desire to improve performance, i.e., increase flow rates in conjunction with narrow molecular weight cut-offs. Membrane thickness remains a barrier to improving performance, as thicker membranes will impede the diffusion of toxins across the membrane as compared to thinner membranes.

In addition to the ability of tailoring the porous structure of membranes, the materials must also be biocompatible when implemented for applications such as hemodialysis. Certain conventional membranes containing thermoplastics have resulted in complications such as cases of thrombocytopenia or increased levels of toxic bisphenol-A in patients.

The Inventors have realized and appreciated that semi-permeable membranes comprising a two-dimensional, atomically thin active layer are particularly effective for filtration applications where a narrow pore-size distribution and high porosity are desirable, such as, for example, dialysis, nanofiltration, diafiltration, and/or forward osmosis. The atomically thin material provides enhanced clearance of certain molecules, such as uremic toxins, due to the thin, nanoscale or sub-nanoscale through-pores that offer little resistance to mass-transfer, while also restricting permeation of larger molecules, such as albumin. With their inherently high permeance, such membranes comprising atomically thin active layers also result in better biocompatibility for applications such as dialysis due to the improved clearance of pro-inflammatory molecules and complement factors. Moreover, the high permeance of the membranes allows for lower blood flow rates during dialysis to achieve adequate target molecule removal, therefore reducing strain on the patient.

The semi-permeable membranes described herein may, in addition to the atomically thin layer, comprise a porous intermediate coating and a porous substrate, wherein the porous intermediate coating is disposed on the atomically thin layer between the atomically thin layer and the porous substrate. While the atomically thin layer is relatively thin and comprises pores with a narrow pore-size distribution, the intermediate coating is comparatively thicker and comprises pores with an average pore size larger than those of the atomically thin layer, and the substrate is the thickest layer of the membrane and comprises yet even larger average pore sizes than both the pores of the atomically thin layer and the intermediate coating. The Inventors have realized and appreciated that configuring the membrane in this way, with an intermediate coating having a thickness and material properties between the two extremes of the atomically thin layer and the substrate material, provides a tailored membrane with better performance, for example, higher permeance and improved clearance of target molecules (e.g., uremic toxins), as compared to conventional membranes. In addition, the intermediate coating may also provide mechanical support for the atomically thin layer and may constitute an additional barrier to prevent unwanted species (e.g., pathogens) from transporting through the membrane. In certain embodiments, the substrate would not have sufficient affinity for the atomically thin layer without the intermediate coating being disposed on the atomically thin layer.

The Inventors have also realized and appreciated that the semi-permeable membrane may be particularly well suited for certain applications, such as dialysis (e.g., hemodialysis), due to the smoothness, biocompatibility, and/or hemocompatibility of the two-dimensional atomically thin layer (e.g., graphene). Specifically, in embodiments where the two-dimensional atomically thin layer is positioned on an external surface of the membrane exposed to a flow of solution to be filtered (e.g., blood or other biological liquids, in some embodiments), the small pore sizes and relatively smooth surface of the atomically thin layer may advantageously reduce, or substantially prevent, fouling as blood and components thereof (e.g., proteins such as albumin) do not readily adhere to the atomically thin layer.

Certain embodiments of dialysis systems comprising a first compartment configured to receive a flow of blood, a second compartment configured to receive a flow of a dialysate, and the semi-permeable membrane disposed between the first compartment and the second compartment, and related methods, are also described herein. Such systems and methods may be used for hemodialysis to facilitate selective transport of, for example, target molecules such as toxins (e.g., uremic toxins) comprising small molecules, small proteins/peptides, and protein-bound uremic toxins (PBUTs), through the semi-permeable membrane, while retaining larger molecules (e.g., albumin). The systems described herein may allow for smaller and more compact dialysis devices while providing a more efficient dialysis mechanism that reduces treatment time and strain on the patient as compared to conventional dialysis technologies.

As described above, the semi-permeable membrane may comprise an atomically thin layer. An atomically thin layer can, for example, be a layer of graphene, which is a one atom thick allotrope of carbon. The theoretical thickness of a sheet of graphene is 0.345 nm, and so an atomically thin layer comprising a single layer of graphene would be expected to have a thickness of approximately 0.345 nm. In some embodiments, an atomically thin layer as described herein may include multiple atomically thin layers (e.g., 2, 5, 10 layers, etc.). Where an atomically thin layer comprises multiple atomically thin layers, layers may be stacked on one another and/or layers may be bonded to adjacent layers, such that the total thickness is the cumulative sum of the thickness of each atomically thin layer. In certain embodiments, when multiple atomically thin layers are grown, they may be bonded to one another as a result of the formation process. These dimensions of an atomically thin layer have particular importance in performing the filtration techniques described herein, since in large part it is the thin nature of these materials that allow high permeance and high flow rates while maintaining better selectivity as compared, for example, conventional polymeric membranes.

As used herein, an “atomically thin layer” refers to a structure formed from one or more planar atomic layers of materials. Atomically thin layers, also known as two-dimensional monolayers or two-dimensional topological materials, are crystalline materials composed of a single layer, or a few layers, of atoms. For example, as described above, a layer of graphene is typically a one atom thick allotrope of carbon, though multiple layers may also be present. Without wishing to be bound by theory, atomically thin materials typically have strong chemical bonds within a plane or layer, but have relatively weaker bonds out of the plane with neighboring planes or layers. Therefore, atomically thin materials typically form sheets of material that may be a single atom thick (i.e., monolayer sheets) to thicker sheets that include several adjacent planes of atoms. For example, an atomically thin layer and/or material may be considered to be a sheet or layer of material including one or more adjacent crystal planes extending parallel to a face of the sheet or layer. An atomically thin material may have a thickness corresponding to any appropriate number of crystal planes including sheets with a thickness corresponding to 1 atomic layer, or in some instances, a thickness that is less than or equal to 2, 3, 4, 5, or 10 atomic layers, or any other appropriate number of atomic layers. In some embodiments, for example, the thickness of the atomically thin layer is between greater than or equal to 0.1 nm and less than or equal to 10 nm. Suitable thicknesses of the atomically thin layer are described in further detail herein. Atomically thin materials may also be referred to as ultra-strength materials and/or two-dimensional (2D) materials as well.

For the sake of clarity, the embodiments and examples described below are primarily directed to an atomically thin layer comprising graphene. However, the membranes, systems, and methods described herein are not so limited and the atomically thin layer may comprise any of a variety of suitable materials. In some embodiments, for example, appropriate atomically thin materials that may be used to form an atomically thin layer include, but are not limited to, hexagonal boron nitride, molybdenum disulfide, vanadium pentoxide, silicon, doped-graphene, graphene oxide, hydrogenated graphene, fluorinated graphene, covalent organic frameworks, layered transition metal dichalcogenides (e.g., MoS₂, TiS₂, etc.), two dimensional oxides (e.g. graphene oxide, NiO₂, etc.), layered Group-IV and Group-III metal chalcogenides (e.g., SnS, PbS, GeS, etc), silicene, germanene, layered binary compounds of Group IV elements and Group III-V elements (e.g., SiC, GeC, SiGe), two-dimensional polymers, two-dimensional covalent organic frameworks, two-dimensional metal organic frameworks, self-assembled two-dimensional networks such as those made using DNA origami, and/or combinations thereof. Other atomically thin materials are also possible.

The atomically thin layer may, in some embodiments, be functionalized and/or coated with one or more materials to improve the biocompatibility and/or selectivity of the atomically thin layer, and/or adhesion of the atomically thin layer to the intermediate coating. Suitable functionalization and/or coating materials include, for example, ethers, amines, carboxyl groups, carbonyl groups, acrylic acid, dextran, and/or combinations thereof. In certain embodiments, a benign polymer (e.g., polyaniline) may be used to functionalize and/or coat the atomically thin layer, which advantageously improves the biocompatibility of the atomically thin layer and reduces its toxicity. In some embodiments, polyethylene glycol (PEG), heparin, zwitterionic molecules, and/or other materials applicable to biomedical devices that improve biocompatibility and/or hemocompatibility may be applied to the atomically thin layer using known methods to functionalize and/or coat such surfaces. Pyrene, derivatives thereof, and/or other polycyclic aromatic hydrocarbons may be used, in some embodiments, to facilitate functionalization of the atomically thin layer and/or promote adhesion of the atomically thin layer to the intermediate coating. The atomically thin layer may be functionalized by plasma treatment, in some embodiments. For example, oxygen plasma may be used to functionalize the surface of the atomically thin layer with oxygen-containing groups. In certain embodiments, the plurality of pores of the atomically thin layer may be formed by plasma treatment.

To distinguish an atomically thin layer, such as a nanoporous atomically thin layer, from an intermediate coating and/or a substrate, in the description herein an atomically thin layer may be referred to as an “active layer.” Together, the atomically thin active layer, intermediate coating, and the substrate form a composite membrane. Nonetheless, the combined effective thickness of such a composite membrane may still be several times smaller than that of conventional membranes. According to certain embodiments, the combined effective thickness of the composite membrane may be greater than that of conventional membranes, but the combined effective thickness of the atomically thin active layer and intermediate coating is smaller than that of conventional membranes, and the substrate may be configured to allow or facilitate fluid flow or mixing. In certain embodiments, for example, the combined effective thickness of the composite membrane may be between about 10 micrometers and about 5 millimeters. In some embodiments, the combined effective thickness of the atomically thin layer and the intermediate coating may be between about 10 nanometers and about 100 micrometers. Suitable thicknesses of the composite membrane are explained in further detail below.

As described herein, the semi-permeable membrane may comprise an intermediate coating disposed on the atomically thin layer, which may be disposed directly on the atomically thin layer, in some embodiments. In certain embodiments, the intermediate coating may be adhered (e.g., bonded) to the atomically thin layer. According to certain embodiments, the intermediate coating at least partially provides structural (e.g., mechanical) stability and/or maintains the structural integrity of at least a portion of the membrane (e.g., the atomically thin layer) during use. In certain embodiments, for example, the porous intermediate coating may be configured to withstand applied operating pressures (i.e., pressure differences across the membrane), while maintaining flexibility and avoiding fracturing during bending. In certain embodiments, for example, the intermediate coating disposed on the atomically thin layer may be capable of withstanding pressures greater than or equal to 30 kPa, greater than or equal to 100 kPa, greater than or equal to 300 kPa, greater than or equal to 1 MPa, or more. In some embodiments, the intermediate coating disposed on the atomically thin layer may be capable of withstanding pressures less than or equal to 10 MPa, less than or equal to 1 MPa, less than or equal to 300 kPa, less than or equal to 100 kPa, or less. In some embodiments, the intermediate coating is porous such that one or more molecules diffusing through the atomically thin layer may then diffuse through the pores of the intermediate coating. In certain embodiments, for example, the intermediate coating has permeance for molecular transport by diffusion and for fluid flow that does not impede mass transfer. In some embodiments, the permeance of the intermediate coating may be greater than the permeance of the atomically thin layer (e.g., two times greater, three times greater, four times greater, five times greater, or more).

The intermediate coating may comprise a plurality of pores having an average pore diameter larger than the pores of the atomically thin layer (e.g., larger than 3.8 nm), but smaller than the pores of the porous substrate (e.g., smaller than 100 micrometers). The thickness of the intermediate coating may, in some embodiments, be thicker than the atomically thin layer (e.g., thicker than 1 nm) but thinner than the substrate (e.g., thinner than 50 micrometers). Suitable pore sizes and thicknesses of the intermediate coating are described in further detail herein.

The porous intermediate coating may comprise any of a variety of suitable materials. In some embodiments, the porous intermediate coating comprises cellulose and/or other polymers or copolymers. Exemplary cellulosic materials include, but are not limited to, cellulose triacetate, cellulose diacetate, and/or cellulose acetate. Exemplary polymers include polyethersulfone, polysulfone, polyacrylonitrile, polyacrylamide, polyamide, polymethylmethacrylate, polyvinylidene fluoride, polyester polymer alloy, and/or ethylene vinyl alcohol copolymer. Other materials are also possible. Certain materials that may be used for dialysis (e.g., hemodialysis), in certain embodiments, include cellulose triacetate, polyethersulfone, polysulfone, polyacrylonitrile, polymethylmethacrylate, and/or ethylene vinyl alcohol copolymer.

According to certain embodiments, the intermediate coating may be modified using any of a variety of suitable additives and/or surface modifiers that improve the biocompatibility and/or wettability of the intermediate coating, and/or adhesion of the intermediate coating to the atomically thin layer. Suitable additives and/or surface modifiers include, for example, PEG and/or zwitterionic molecules which may improve the water retention properties of the semi-permeable membrane. In some embodiments, an epoxy or other material may be used as a surface modifier to plug any surface defects in the intermediate coating. Other materials may be employed to functionalize and/or coat the intermediate coating to improve its biocompatibility and/or hemocompatibility by increasing the capacity of the intermediate coating to adsorb complement factors and other molecules.

The semi-permeable membrane may comprise a substrate, wherein the substrate is configured such that the intermediate coating is disposed between the atomically thin layer and the substrate. According to certain embodiments, the substrate may be adhered (e.g., bonded) to the intermediate coating. The substrate may, in some embodiments, advantageously at least partially provide structural stability and/or maintain the structural integrity of the membrane (e.g., the atomically thin layer and the intermediate coating) during use. According to some embodiments, the substrate is porous such that one or more molecules diffusing through the atomically thin layer and the pores of the intermediate coating may then diffuse through the porous substrate.

The porous substrate may comprise a plurality of pores having an average pore diameter larger than the pores of the atomically thin layer and the porous intermediate coating (e.g., larger than 200 nm). In certain embodiments, the porous substrate has a thickness larger than the thickness of the atomically thin layer and the porous intermediate (e.g., larger than 3 micrometers). Suitable pore sizes and thicknesses of the porous substrate are described in further detail herein.

The porous substrate may comprise any of a variety of suitable materials. According to certain embodiments, the porous substrate may be at least partially hydrophilic. In some embodiments, for example, the porous substrate comprises a polymer, a ceramic, a metal, and/or combinations thereof. Exemplary materials include, but are not limited to, nylon, polyethersulfone, polysulfone, polylactic acid, polyvinyl chloride, polyethylene, polypropylene, polymethyl methacrylate, titanium, titania, alumina, silica, glass, silicon nitride, silicone, and/or silicon. Other materials are also possible. Additionally, in some instances, the porous substrate may include a mixture of materials and/or multiple layers of materials. For example, the substrate may comprise a polymer layer disposed on a metal and/or ceramic layer, according to some embodiments, or polyethersulfone coated on glass. The substrate may be functionalized and/or coated with one or more materials that improve the biocompatibility and/or hemocompatibility of the substrate.

Turning now to the figures, specific non-limiting embodiments are described in more detail. It should be understood that various features of the separately described embodiments may be used together as the current disclosure is not limited to the specific embodiments depicted in the figures and described below.

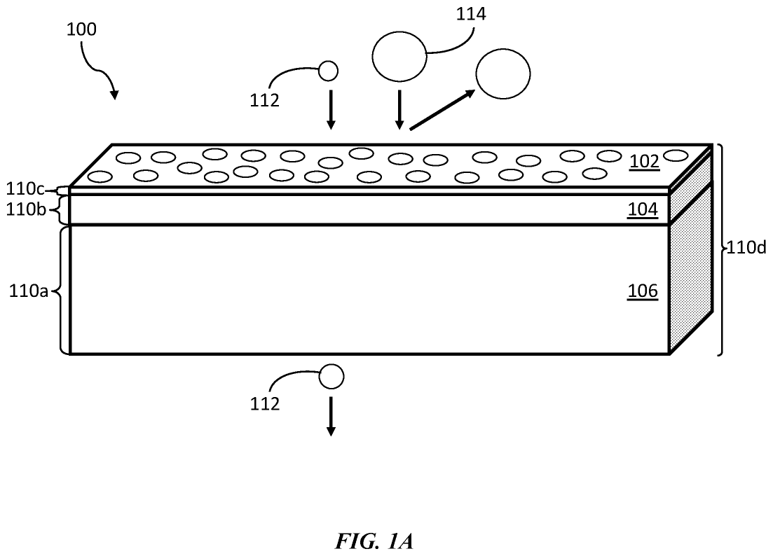

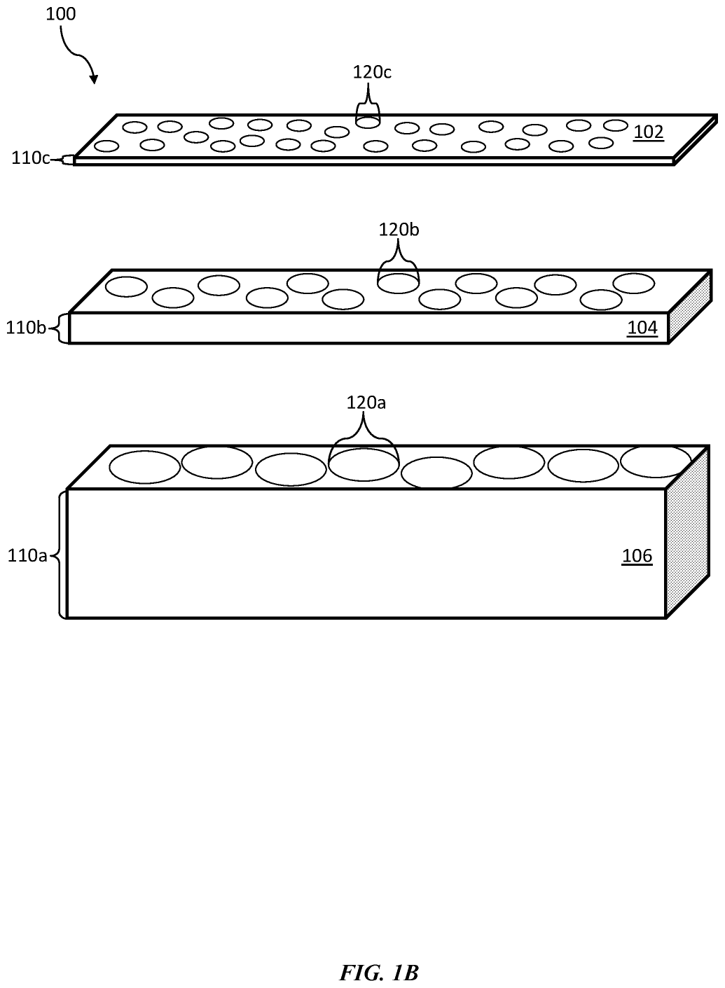

FIG. 1A shows, according to some embodiments, a schematic diagram of a semi-permeable membrane, and FIG. 1B shows, an expanded schematic diagram of the semi-permeable membrane shown in FIG. 1A. In the example of FIG. 1A, membrane 100 comprises atomically thin layer 102, intermediate coating 104 disposed on atomically thin layer 102, and substrate 106, wherein intermediate coating 104 is disposed between atomically thin layer 102 and substrate 106. Methods of fabricating membrane 100 are explained in greater detail herein.

Although FIG. 1A shows a single atomically thin active layer, the composite membrane may comprise more than one such active layer, as discussed above, as the disclosure is not so limited in this regard. In some such embodiments, the pores are aligned in the stacked atomically thin active layers such that they pass from an external surface of an outermost atomically thin active layer oriented away from the substrate to an opposing surface of an innermost atomically thin active layer oriented towards the adjacent substrate thus providing fluid communication between opposing surfaces of the active layer.

Referring to FIG. 1B, atomically thin layer 102, intermediate coating 104, and substrate 106 may each comprise a plurality of pores. The plurality of pores between each layer of membrane 100 may be configured, in some embodiments, such that there is fluid communication between the outer surface of atomically thin layer 102 and the opposing outer surface of substrate 106. The fluid communication between atomically thin layer 102 and substrate 106 may advantageously allow a desired target species to diffuse through membrane 100 during use.

In certain embodiments, the plurality of pores of atomically thin layer 102, intermediate coating 104, and substrate 106 may be open pores (e.g., channels) that allow for the diffusion of a target species through membrane 100. According to some embodiments, for example, the plurality of pores may extend directly through the membrane from the outer surface of atomically thin layer 102 to the opposing outer surface of substrate 106. In certain embodiments, atomically thin layer 102, intermediate coating 104, and substrate 106 may be configured such that at least a portion of the plurality of pores of each layer are interconnected such that a target species may flow (e.g., diffuse) through each layer of membrane 100 as desired. In certain embodiments, the plurality of pores (e.g., open pores) of atomically thin layer 102, intermediate coating 104, and substrate 106 may not be substantially filled with other materials. Suitable pore sizes and porosities of each layer are described below in greater detail. In some embodiments, the pore sizes and/or porosities may be determined directly, for example, using spectroscopy (e.g., Raman spectroscopy), imaging (e.g., scanning electron microscopy, aberration-corrected scanning transmission electron microscopy), or another suitable technique. In certain embodiments, the pore sizes and/or porosities may be measured indirectly, such as, for example, testing diffusion through the membrane with various species of known sizes, mercury porosimetry, or testing fluid flow through the membrane at various pressures.

In some embodiments, since the active layer is atomically thin, resistance to flow can be much lower than that of other typical membranes, resulting in a much higher permeability. Referring, for example, to FIGS. 1A and 1B, the thickness of atomically thin layer 102 may be average thickness 110c. In some embodiments, atomically thin layer 102 has an average thickness 110c greater than or equal to 0.1 nm, greater than or equal to 0.2 nm, greater than or equal to 0.3 nm, greater than or equal to 0.4 nm, greater than or equal to 0.5 nm, greater than or equal to 0.6 nm, greater than or equal to 0.7 nm, greater than or equal to 0.8 nm, greater than or equal to 0.9 nm, greater than or equal to 1 nm, greater than or equal to 2 nm, or more. In certain embodiments, atomically thin layer 102 has an average thickness 110c less than or equal to 5 nm, less than or equal to 2 nm, less than or equal to 1 nm, less than or equal to 0.9 nm, less than or equal to 0.8 nm, less than or equal to 0.7 nm, less than or equal to 0.6 nm, less than or equal to 0.5 nm, less than or equal to 0.4 nm, less than or equal to 0.3 nm, less than or equal to 0.2 nm, or less. Combinations of the above recited ranges are also possible (e.g., the atomically thin layer has an average thickness between greater than or equal to 0.1 nm and less than or equal to 5 nm, the atomically thin layer has an average thickness between greater than or equal to 0.4 nm and less than or equal to 0.6 nm). Other ranges are also possible.

In certain embodiments wherein the composite membrane comprises more than one atomically thin layer (e.g., 2, 3, 4, 5, or 10 atomically thin layers), the overall average thickness may be greater than or equal to 1 nm, greater than or equal to 2 nm, greater than or equal to 3 nm, greater than or equal to 4 nm, greater than or equal to 5 nm, greater than or equal to 10 nm, greater than or equal to 20 nm, or greater. In some embodiments, the overall average thickness of the atomically thin layer may be less than or equal to 50 nm, less than or equal to 20 nm, less than or equal to 10 nm, less than or equal to 5 nm, less than or equal to 4 nm, less than or equal to 3 nm, less than or equal to 2 nm, or less. Combinations of the above recited ranges are also possible (e.g., the overall average thickness of the atomically thin layer may be greater than or equal to 1 nm and less than or equal to 50 nm, the overall thickness of the atomically thin layer may be greater than or equal to 5 nm and less than or equal to 10 nm). Other ranges are also possible.

In some embodiments, the pores of atomically thin layer 102 may be uniform. In other embodiments, the pores of atomically thin layer 102 may be asymmetric, such that the pores on the surface of atomically thin layer 102 that is exposed to fluid (e.g., during dialysis) have a relatively smaller average pore size, while the pores on the opposite surface of atomically thin layer 102 (e.g., facing intermediate coating 104) have a relatively larger average pore size. Such a configuration may, in some embodiments, advantageously provide a barrier to prevent unwanted pathogens (e.g., bacteria and viruses) from transporting through the membrane (e.g., from the dialysate side to the blood side during hemodialysis). Methods of forming the asymmetric pores are explained in greater detail herein.

The size and density of the plurality of pores in the atomically thin active layer may be optimized for a particular application and the sizes of target molecules, ions, particles, or other filtrate species that are intended to pass through or be inhibited by the atomically thin active layer. According to certain embodiments, the pore size of the plurality of pores of atomically thin layer 102 may be any of a variety of suitable average pore sizes 120c (e.g., mean pore diameters), as shown in FIGS. 1A and 1B. In some embodiments, for example, atomically thin layer 102 comprises a plurality of pores having an average pore size 120c (e.g., mean pore diameter) greater than or equal to 0.3 nm, greater than or equal to 0.6 nm, greater than or equal to 1 nm, greater than or equal to 1.5 nm, greater than or equal to 2.0 nm, greater than or equal to 2.5 nm, greater than or equal to 3 nm, greater than or equal to 3.5 nm, or more. In some embodiments, atomically thin layer 102 comprises a plurality of pores having an average pore size 120c (e.g., mean pore diameter) less than or equal to 3.8 nm, less than or equal to 3.5 nm, less than or equal to 3 nm, less than or equal to 2.5 nm, less than or equal to 2 nm, less than or equal to 1.5 nm, less than or equal to 1 nm, less than or equal to 0.6 nm, or less. Combinations of the above recited ranges are also possible (e.g., the atomically thin layer comprises a plurality of pores having an average pore size between greater than or equal to 0.3 nm and less than or equal to 3.8 nm, the atomically thin layer comprises a plurality of pores having an average pore size between greater than or equal to 2 nm and less than or equal to 3 nm). Other ranges are also possible.

Without wishing to be bound by theory, the plurality of pores of the atomically thin layer may have an average pore size (e.g., mean pore diameter) less than or equal to the size of a target molecule to be restricted from transporting through the membrane. For example, the plurality of pores of the atomically thin layer may have an average pore size less than or equal to 3.8 nm, according to certain non-limiting embodiments, to restrict certain target molecules with an average characteristic dimension larger than 3.8 nm (e.g., albumin) from diffusing through the membrane.

According to some embodiments, the atomically thin layer may be porous to advantageously facilitate high permeance and selective transport of target molecules, ions, particles, or other filtrate species. According to some embodiments, atomically thin layer 102 may have a relatively low percent porosity as compared to intermediate coating 104 and/or substrate 106. In some embodiments, for example, the percent porosity of atomically thin layer 102 is greater than or equal to 0.01%, greater than or equal to 0.1%, greater than or equal to 1%, greater than or equal to 5%, greater than or equal to 10%, greater than or equal to 15%, greater than or equal to 20%, greater than or equal to 25%, or more. In some embodiments, the percent porosity of atomically thin layer 102 is less than or equal to 30%, less than or equal to 25%, less than or equal to 20%, less than or equal to 15%, less than or equal to 10%, less than or equal to 5%, less than or equal to 1%, less than or equal to 0.1%, or less. Combinations of the above recited ranges are also possible (e.g., the percent porosity of atomically thin layer 102 is between greater than or equal to 0.01% and less than or equal to 30%, the porosity of atomically thin layer 102 is between greater than or equal to 10% and less than or equal to 20%). Other ranges are also possible.

It should be understood that, in some embodiments, one or more properties of atomically thin layer 102 (e.g., thickness, average pore size, and/or porosity) may vary across atomically thin layer 102. For example, in certain embodiments, atomically thin layer 102 may have a first porosity between greater than or equal to 0.01% and less than or equal to 10% across a first portion of atomically thin layer 102 and a second porosity between greater than or equal to 20% and less than or equal to 30% across a second portion of atomically thin layer 102. Gradients in the thickness and the average pore size are also possible across atomically thin layer 102, in some embodiments.

Referring to FIG. 1A, membrane 100 may comprise intermediate coating 104 disposed on atomically thin layer 102. Methods of disposing intermediate coating 104 on atomically thin layer 102 are described herein in greater detail.

The porous intermediate coating may have any of a variety of suitable thicknesses. As described herein, the thickness of the porous intermediate coating may be greater than the thickness of the atomically thin layer, which advantageously provides, for example, structural (e.g., mechanical) stability to the membrane (e.g., atomically thin layer) and/or improved durability of the membrane after repeated use. Referring to FIGS. 1A and 1B, for example, the thickness of intermediate coating 104 may be average thickness 110b. In certain embodiments, porous intermediate coating 104 has an average thickness 110b greater than or equal to 10 nm, greater than or equal to 50 nm, greater than or equal to 100 nm, greater than or equal to 1 micrometer, greater than or equal to 5 micrometers, greater than or equal to 10 micrometers, greater than or equal to 50 micrometers, or more. In some embodiments, porous intermediate coating 104 has an average thickness 110b less than or equal to 100 micrometers, less than or equal to 50 micrometers, less than or equal to 10 micrometers, less than or equal to 5 micrometers, less than or equal to 1 micrometer, less than or equal to 100 nm, less than or equal to 50 nanometers, or less. Combinations of the above recited ranges are also possible (e.g., the porous intermediate coating has an average thickness between greater than or equal to 10 nm and less than or equal to 100 micrometers, the porous intermediate coating has an average thickness between greater than or equal to 1 micrometer and less than or equal to 3 micrometers). Other ranges are also possible.

As depicted in FIG. 1A, intermediate coating 104 may be configured to facilitate transport of target molecules (e.g., first species 112) through membrane 100. Accordingly, as described herein, the porous intermediate coating may comprise a plurality of pores having pore sizes greater than the pores of the atomically thin layer (e.g., greater than 3.8 nm). According to some embodiments, the pore size of the plurality of pores of intermediate coating 104 may be any of a variety of suitable average pore sizes 120b (e.g., mean pore diameters), as shown in FIG. 1B. In certain embodiments, for example, intermediate coating 104 comprises a plurality of pores having an average pore size 120b (e.g., mean pore diameter) greater than or equal to 10 nm, greater than or equal to 20 nm, greater than or equal to 50 nm, greater than or equal to 100 nm, greater than or equal to 150 nm, greater than or equal to 200 nm, greater than or equal to 250 nm, greater than or equal to 500 nm, or more. In some embodiments, intermediate coating 104 comprises a plurality of pores having an average pore size 120b (e.g., mean pore diameter) less than or equal to 1 micron, less than or equal to 500 nm, less than or equal to 250 nm, less than or equal to 200 nm, less than or equal to 150 nm, less than or equal to 100 nm, less than or equal to 50 nm, less than or equal to 20 nm, or less. Combinations of the above recited ranges are also possible (e.g., the porous intermediate coating comprises a plurality of pores having an average pore size between greater than or equal to 10 nm and less than or equal to 1 micron, the porous intermediate coating comprises a plurality of pores having an average pore size between greater than or equal to 100 nm and less than or equal to 150 nm). Other ranges are also possible.

In some embodiments, the pores of intermediate coating 104 may be uniform. In other embodiments, the pores of the intermediate coating 104 may be asymmetric, such that the pores on the surface of intermediate coating 104 disposed on atomically thin layer 102 have a relatively smaller average pore size, while the pores on the opposite surface of intermediate coating 104 (e.g., facing substrate 106) have a relatively larger average pore size. Such a configuration may, in some embodiments, provide mechanical support for the atomically thin layer and/or promote adhesion of the intermediate coating to the atomically thin layer. In yet other embodiments, the pores of the intermediate coating 104 may be asymmetric, such that the pores on the surface of intermediate coating 104 disposed on atomically thin layer 102 have a relatively larger average pore size, while the pores on the opposite surface of intermediate coating 104 (e.g., facing substrate 106) have a relatively smaller average pore size.

According to certain embodiments, the relatively smaller pores on the surface of intermediate coating 104 oriented towards and disposed against atomically thin layer 102 may, in addition to providing mechanical support to the atomically thin layer 102, also advantageously provide a second barrier (in addition to atomically thin layer 102) to prevent unwanted pathogens (e.g., bacteria and viruses) from transporting through the membrane (e.g., from the dialysate side to the blood side during hemodialysis). Methods of forming the asymmetric pores are explained in greater detail herein.

According to certain embodiments, the pores on the surface of intermediate coating 104 disposed on atomically thin layer 102 may have an average pore size greater than or equal to 10 nm, greater than or equal to 20 nm, greater than or equal to 30 nm, greater than or equal to 40 nm, or more. In some embodiments, the pores on the surface of intermediate coating 104 disposed on atomically thin layer 102 have an average pore size less than or equal to 50 nm, less than or equal to 40 nm, less than or equal to 30 nm, less than or equal to 20 nm, or less. Combinations of the above recited ranges are also possible (e.g., the pores on the surface of intermediate coating 104 disposed on atomically thin layer 102 have an average pore size greater than or equal to 10 nm and less than or equal to 50 nm, the pores on the surface of intermediate coating 104 disposed on atomically thin layer 102 have an average pore size greater than or equal to 20 nm and less than or equal to 40 nm). Other ranges are also possible.

In some embodiments, the pores on the surface of intermediate coating 104 oriented towards substrate 106 (e.g., the surface that is opposite the surface of intermediate coating 104 disposed on atomically thin layer 102) may have an average pore size greater than or equal to 100 nm, greater than or equal to 150 nm, greater than or equal to 200 nm, greater than or equal to 250 nm, greater than or equal to 500 nm, or more. In certain embodiments, the pores on the surface of intermediate coating 104 facing substrate 106 have an average pore size less than or equal to less than or equal to 1 micron, less than or equal to 500 nm, less than or equal to 250 nm, less than or equal to 200 nm, less than or equal to 150 nm, or less. Combinations of the above recited ranges are also possible (e.g., the pores on the surface of intermediate coating 104 facing substrate 106 have an average pore size greater than or equal to 100 nm and less than or equal to 1 micron, the pores on the surface of intermediate coating 104 facing substrate 106 have an average pore size greater than or equal to 150 nm and less than or equal to 250 nm). Other ranges are also possible.

According to certain embodiments, the percentage change between the average size of the pores on the surface of intermediate coating 104 disposed on atomically thin layer 102 compared to the pores on the surface of intermediate coating 104 facing substrate 106 may be greater than or equal to 100%, greater than or equal to 500%, greater than or equal to 1000%, greater than or equal to 2000%, or more. In some embodiments, the percentage change between the average size of the pores on the surface of intermediate coating 104 disposed on atomically thin layer 102 compared to the pores on the surface of intermediate coating 104 facing substrate 106 may be less than or equal to 3000%, less than or equal to 2000%, less than or equal to 1000%, less than or equal to 500%, or less. Combinations of the above recited ranges are also possible (e.g., the percentage change between the average size of the pores on the surface of intermediate coating 104 disposed on atomically thin layer 102 compared to the pores on the surface of intermediate coating 104 facing substrate 106 may be greater than or equal to 100% and less than or equal to 3000%, the percentage change between the average size of the pores on the surface of intermediate coating 104 disposed on atomically thin layer 102 compared to the pores on the surface of intermediate coating 104 facing substrate 106 may be greater than or equal to 1000% and less than or equal to 2000%). Other ranges are also possible.

According to some embodiments, the intermediate coating may be at least partially porous to advantageously facilitate the permeance and transport of target species that have diffused through the atomically thin layer. In certain embodiments, the percent porosity of intermediate coating 104 may be greater than atomically thin layer 102 and less than porous substrate 106. In some embodiments, for example, the percent porosity of intermediate coating 104 is greater than or equal to 5%, greater than or equal to 10%, greater than or equal to 20%, greater than or equal to 30%, greater than or equal to 40%, greater than or equal to 50%, greater than or equal to 60%, greater than or equal to 70%, or more. In certain embodiments, the percent porosity of intermediate coating 104 is less than or equal to 80%, less than or equal to 70%, less than or equal to 60%, less than or equal to 50%, less than or equal to 40%, less than or equal to 30%, less than or equal to 20%, less than or equal to 10%. or less. Combinations of the above recited ranges are also possible (e.g., the percent porosity of the intermediate coating is between greater than or equal to 5% and less than or equal to 80%, the percent porosity of the intermediate coating is between greater than or equal to 30% and less than or equal to 60%). Other ranges are also possible.

It should be understood that, in some embodiments, one or more properties of intermediate coating 104 (e.g., thickness, average pore size, and/or porosity) may vary across intermediate coating 104. For example, in certain embodiments, intermediate coating 104 may have a first porosity between greater than or equal to 5% and less than or equal to 40% across a first portion of intermediate coating 104 and a second porosity between greater than or equal to 50% and less than or equal to 80% across a second portion of intermediate coating 104. Gradients in the thickness and the average pore size are also possible across intermediate coating 104, in some embodiments.

Referring again to FIG. 1A, membrane 100 may comprise substrate 106, wherein substrate 106 is configured such that intermediate coating 104 is disposed between atomically thin layer 102 and substrate 106. Methods of fabricating the structure of membrane 100 in this way are described herein in greater detail.

The porous substrate may have any of a variety of suitable thicknesses. As described herein, the thickness of the porous substrate may be greater than the thickness of the atomically thin layer and the intermediate coating, which advantageously provides mechanical stability to the membrane. Referring to FIGS. 1A and 1B, for example, the thickness of substrate 106 may be average thickness 110a. According to some embodiments substrate 106 has an average thickness 110a greater than or equal to 10 micrometers, greater than or equal to 50 micrometers, greater than or equal to 100 micrometers, greater than or equal to 200 micrometers, greater than or equal to 500 micrometers, greater than or equal to 1 millimeter, greater than or equal to 2 millimeters, or more. In certain embodiments, substrate 106 has an average thickness 110a less than or equal to 5 millimeters, less than or equal to 2 millimeters, less than or equal to 1 millimeter, less than or equal to 500 micrometers, less than or equal to 200 micrometers, less than or equal to 100 micrometers, less than or equal to 50 micrometers, or less. Combinations of the above recited ranges are also possible (e.g., the porous substrate has an average thickness between greater than or equal to 10 micrometers and less than or equal to 5 millimeters, the porous substrate has an average thickness between greater than or equal to 200 micrometers and less than or equal to 500 micrometers). Other ranges are also possible.

As depicted in FIG. 1A, substrate 106 may be configured to facilitate transport of target molecules (e.g., first species 112) through membrane 100. Accordingly, as described herein, the porous substrate may comprise a plurality of pores having pore sizes greater than the pores of the atomically thin layer and the intermediate coating (e.g., greater than 200 nm). In certain embodiments, the pore size of the plurality of pores of substrate 106 may be any of a variety of suitable average pore sizes 120a (e.g., mean pore diameters), as shown in FIG. 1B. In some embodiments, for example, substrate 106 comprises a plurality of pores having an average pore size 110a (e.g., mean pore diameter) greater than or equal to 1 micrometer, greater than or equal to 5 micrometers, greater than or equal to 10 micrometers, greater than or equal to 50 micrometers, greater than or equal to 100 micrometers, greater than or equal to 200 micrometers, greater than or equal to 300 micrometers, greater than or equal to 400 micrometers, or more. In certain embodiments, substrate 106 comprises a plurality of pores having an average pore size 110a (e.g., mean pore diameter) less than or equal to 500 micrometers, less than or equal to 400 micrometers, less than or equal to 300 micrometers, less than or equal to 200 micrometers, less than or equal to 100 micrometers, less than or equal to 50 micrometers, less than or equal to 10 micrometers, less than or equal to 5 micrometers, or less. Combinations of the above recited ranges are also possible (e.g., the porous substrate comprises a plurality of pores having an average pore size between greater than or equal to 100 micrometers and less than or equal to 500 micrometers, the porous substrate comprises a plurality of pores having an average pore size between greater than or equal to 200 nm and less than or equal to 400 nm). Other ranges are also possible.

In some embodiments, the pores of substrate 106 may be uniform. In other embodiments, the pores of substrate 106 may be asymmetric, such that the pores on the surface of substrate 106 facing intermediate coating 104 have a relatively smaller average pore size, while the pores on the opposite surface of substrate 106 have a relatively larger average pore size. Such a configuration may, in some embodiments, advantageously provide a third barrier (in addition to atomically thin layer 102 and intermediate coating 104) to prevent unwanted pathogens (e.g., bacteria and viruses) from transporting through the membrane (e.g., from the dialysate side to the blood side during hemodialysis). Methods of forming the asymmetric pores are explained in greater detail herein.

In certain embodiments, the substrate may be at least partially porous to advantageously facilitate the permeance and transport of target species that have diffused through the atomically thin layer and the porous intermediate coating. According to some embodiments, the percent porosity of substrate 106 may be greater than atomically thin layer 102 and intermediate coating 104. In certain embodiments, for example, the percent porosity of substrate 106 is greater than or equal to 20%, greater than or equal to 30%, greater than or equal to 40%, greater than or equal to 50%, greater than or equal to 60%, greater than or equal to 70%, greater than or equal to 80%, or more. In some embodiments, the percent porosity of substrate 106 is less than or equal to 90%, less than or equal to 80%, less than or equal to 70%, less than or equal to 60%, less than or equal to 50%, less than or equal to 40%, less than or equal to 30%, or less. Combinations of the above recited ranges are also possible (e.g., the percent porosity of the substrate is between greater than or equal to 20% and less than or equal to 90%, the percent porosity of the substrate is between greater than or equal to 80% and less than or equal to 90%). Other ranges are also possible.

It should be understood that, in some embodiments, one or more properties of substrate 106 (e.g., thickness, average pore size, and/or porosity) may vary across substrate 106. For example, in certain embodiments, substrate 106 may have a first porosity between greater than or equal to 20% and less than or equal to 50% across a first portion of substrate 106 and a second porosity between greater than or equal to 60% and less than or equal to 90% across a second portion of substrate 106. Gradients in the thickness and the average pore size are also possible across substrate 106, in some embodiments.

In certain embodiments, the pores on the surface of the substrate contacting the intermediate coating may be beveled. According to some embodiments, the surface of the substrate contacting the intermediate coating may be tapered. Such a configuration advantageously maximizes the fraction of pores of the intermediate coating that allow for diffusion of target species (i.e., by minimizing blockage of the pores of the intermediate coating).

The membrane may have any of a variety of suitable combined effective thicknesses. Referring, for example, to FIG. 1A, overall average thickness 110d of membrane 100 may be greater than or equal to 10 micrometers, greater than or equal to 20 micrometers, greater than or equal to 50 micrometers, greater than or equal to 100 micrometers, greater than or equal to 200 micrometers, greater than or equal to 500 micrometers, greater than or equal to 1 millimeter, greater than or equal to 2 millimeters, or more. In some embodiments, overall average thickness 110d of membrane 100 may be less than or equal to 5 millimeters, less than or equal to 2 millimeters, less than or equal to 1 millimeter, less than or equal to 500 micrometers, less than or equal to 200 micrometers, less than or equal to 100 micrometers, less than or equal to 50 micrometers, less than or equal to 20 micrometers, or less. Combinations of the above recited ranges are also possible (e.g., the membrane has an overall average thickness greater than or equal to 10 micrometers and less than or equal to 5 millimeters, the membrane has an overall average thickness greater than or equal to 100 micrometers and less than or equal to 200 micrometers). Combinations of the above recited ranges are also possible.

The membrane may have any of a variety of suitable areas. In some embodiments, for example, the membrane has an area between greater than or equal to 0.25 cm² and less than or equal to 9 cm².

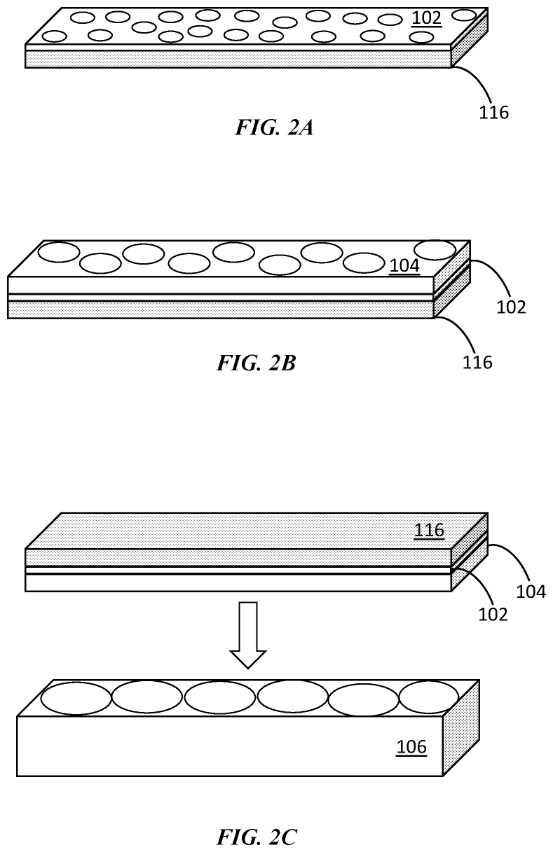

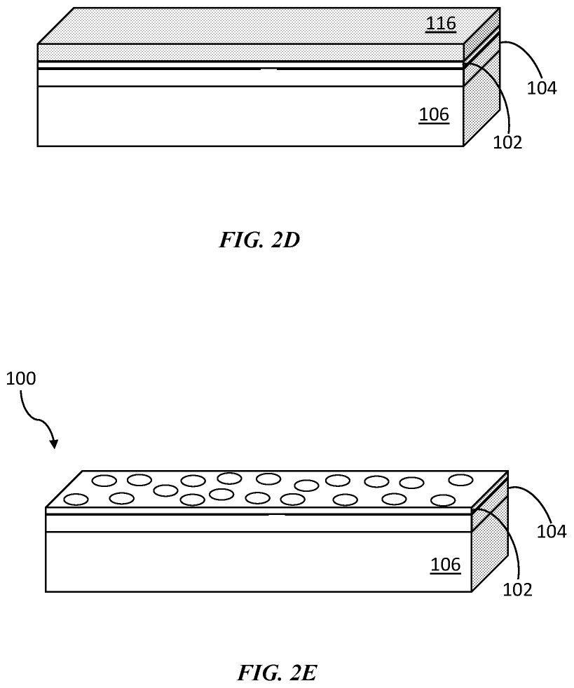

According to certain embodiments, methods of fabricating a composite membrane are described herein. FIGS. 2A-2E show, according to some embodiments, a method of fabricating a semi-permeable membrane. Referring to FIG. 2A, atomically thin layer 102 may be provided on support layer 116 by any of a variety of suitable means, including, but not limited to, synthesizing and/or depositing atomically thin layer 102 on support layer 116. In certain embodiments, the plurality of pores of the atomically thin layer may be formed by low-temperature synthesis of the atomically thin layer, plasma etching, oxidative species etching, and/or catalyzed gas-solid reactions. In certain embodiments, support layer 116 may comprise a metal (e.g., copper). The metal may, in some embodiments, be coated with one or more materials to improve the biocompatibility of the metal.

Referring to FIG. 2B, intermediate coating 104 may be disposed on atomically thin layer 102. According to some embodiments, the intermediate coating disposed directly on the atomically thin layer advantageously avoids the possibility introducing defects into the atomically thin layer. Unnecessary transfer of the atomically thin layer from one substrate to another is avoided, therefore reducing the chance of damaging the atomically thin layer, as the intermediate coating is disposed (e.g., grown) directly on the atomically thin layer. Any of a variety of suitable means may be used to dispose intermediate coating 104 on atomically thin layer 102. In some embodiments, for example, intermediate coating 104 may be disposed onto atomically thin layer 102 by coating (e.g., spin coating, etc.), spraying (electrospraying, direct spraying, etc.), depositing (e.g., vapor deposition such as chemical vapor deposition), casting (e.g., spin casting), and the like. Intermediate coating 104, in some embodiments, may be formed by polymerization (e.g., surface-initiated polymerization, vapor-phase polymerization, interfacial polymerization). Intermediate coating 104 may also advantageously protect atomically thin layer 102 from defect introduction during assembly of the semi-permeable membrane, for example, when the composite structure of the intermediate coating 102 and atomically thin layer 102 are disposed onto substrate 106, which is explained in further detail below.

In certain embodiments, as described above, intermediate coating 104 may include one or more additives and/or surface modifiers that improve certain properties of intermediate coating 104, such as the biocompatibility, wettability, and/or adhesion to the atomically thin layer. In some such embodiments, the one or more additives and/or surface modifiers may be blended with the intermediate coating material prior to disposing intermediate coating 104 on atomically thin layer 102. In certain embodiments, the one or more additives and/or surface modifiers may be disposed on intermediate coating 104 after disposing intermediate coating 104 on atomically thing layer 102. Examples of such additives and/or surface modifiers are described above in greater detail.

The plurality of pores of intermediate coating 104 may be formed by any of a variety of suitable means. According to some embodiments, for example, the plurality of pores are formed by a phase inversion process. In certain embodiments, forming pores in the intermediate coating via a phase inversion process advantageously limits the number of defects introduced in both intermediate coating 104 and atomically thin layer 102, as intermediate coating 104 is configured to protect atomically thin layer 102. In certain embodiments, a polymer solution (e.g., polyethersulfone in N-methyl pyrrolidone) may be immersed in a non-solvent (e.g., water, alcohol, combinations thereof). The concentration of the polymer in solution may be less than or equal to 25 wt. %, less than or equal to 20 wt. %. less than or equal to 15 wt. %, less than or equal to 10 wt. %, less than or equal to 5 wt. %, or less. The composition of polymer, solvent, and anti-solvent (and concentration of polymer) may be chosen such that the average pore size and/or thickness of the of porous coating may be controlled and/or tailored as desired. In certain non-limiting embodiments, for example, an alcohol (e.g., isopropyl alcohol), may added to the polymer solution to promote wetting of the atomically thin layer and modulate polymer precipitation during phase inversion, thereby creating a coating or more uniform thickness with controlled pore characteristics. In other non-limiting embodiments, the polymer concentration may be decreased to enhance porosity, or a milder anti-solvent mixture may be used to allow faster pore development, resulting in larger pore sizes during precipitation. In yet another non-limiting embodiment, a volatile solvent (e.g., methanol) may be used as an additive in the casting solution to modulate pore size (e.g., to provide larger pores). The volatile solvent may, for example, modulate (e.g., decrease) the viscosity of the casting solution, and as the volatile solvent evaporates, the viscosity of the casting solution increases, resulting in a more uniform and stable coating. In other non-limiting embodiments, the antisolvent exchange process is slowed in order to promote an intermediate coating with larger thicknesses. In some embodiments, the plurality of pores is formed and/or treated after the phase inversion process by annealing and/or sintering intermediate coating 104.

In some embodiments, forming the plurality of pores of intermediate coating 104 by phase inversion may advantageously provide asymmetric pores wherein the pores on the surface of intermediate coating 104 disposed on atomically thin layer 102 have a relatively smaller average pore size, while the pores on the opposite surface of intermediate coating 104 (e.g., facing substrate 106) have a relatively larger average pore size. In certain non-limiting embodiments, for example, the antisolvent exchange process may be tailored such that pores on the surface of intermediate coating 104 disposed on atomically thin layer 102 have a smaller average size than the pores on the opposite surface of intermediate coating 104. Phase inversion processes may also be used to form asymmetric pores in atomically thin layer 102 and substrate 106, as the disclosure is not meant to be limiting in this regard.

Referring to FIG. 2C, substrate 106 may be provided by any of a variety of suitable means. In certain embodiments, substrate 106 is printed by, for example, three-dimensional printing (3D-printing) or other additive manufacturing techniques. As described herein, substrate 106 may comprise a mixture of materials and/or multiple layers, which may, for example, be advantageously imparted by 3D-printing. The plurality of pores of substrate 106 may be formed by any of a variety of suitable means, including, but not limited to, 3D-printing substrate 106.

Referring to FIGS. 2C and 2D, intermediate coating 102 disposed on atomically thin layer 102 provided on support layer 116 may be disposed (e.g., compressed) on substrate 106. An adhesion material (e.g., polydimethylsiloxane or other appropriate material) may be coated on substrate 106, in some embodiments, to promote adhesion between substrate 106 and intermediate coating 102. In certain embodiments, any residual solvent left on intermediate coating 104 may assist in adhering intermediate coating 104 to substrate 106 by softening intermediate coating 104. Support layer 116 may then be removed (e.g., etched or peeled away), as shown in FIG. 2E, resulting in semi-permeable membrane 100. In some embodiments, although not shown in the figures, support layer 116 may be removed prior to disposing intermediate coating 102 onto atomically thin layer 102 onto substrate 106. Alternatively, substrate 106 may be printed (e.g., 3D-printed) directly onto a surface of the intermediate coating 102 opposite from the atomically thin layer 102 (e.g., with or without support layer 116), in certain embodiments. In certain embodiments, substrate 106 may be formed by phase inversion or other fabrication processes, such as those described above for forming the intermediate coating (e.g., coating, spraying, depositing, casting, and the like). According to some embodiments, membrane 100 may be annealed to promote adhesion between atomically thin layer 102 and intermediate coating 104 and between substrate 106 and intermediate coating 104.

According to certain embodiments, the anti-solvent used in the phase inversion process to form the plurality of pores of intermediate coating 104 may be loaded onto and/or soaked into substrate 106. In some such embodiments, the phase inversion process occurs as intermediate coating 102 disposed on atomically thin layer 102 provided on support layer 116 is disposed (e.g., compressed) on substrate 106.

The resulting membranes may be applied to any number of different applications. In certain embodiments, the membranes may be employed in a diffusion-based filtration application or in a diafiltration application. In some embodiments related to diffusion-based filtration or diafiltration, solutions and/or gases disposed on either side of the filtration membrane may be flowed, agitated, stirred, or otherwise mixed to help reduce the presence of concentration gradients which may slow a diffusive filtration process. However, embodiments in which one or more solutions and/or gases located adjacent to a filtration membrane are not mixed are also contemplated.

According to some embodiments, the membranes described herein may be used for dialysis (e.g., hemodialysis). Certain other commercial applications of the described membranes include, but are not limited to: water purification to remove pathogens, organic molecules, and salts (desalination/softening); desalting of proteins; portable water filters;

preconcentrators for liquid or gas samples for use in sensing applications; gas separation in energy applications such as natural gas separation (methane from carbon dioxide, hydrogen sulfide, and heavier hydrocarbons) and carbon sequestration; medical implants for allowing only select molecules to go through (e.g., for sensor applications); separation of excess reactants from a reaction mixture; medical implants that allow only select molecules to pass through a membrane (e.g., for sensor applications); controlled drug release devices; and use in fuel cells as proton-selective membranes.

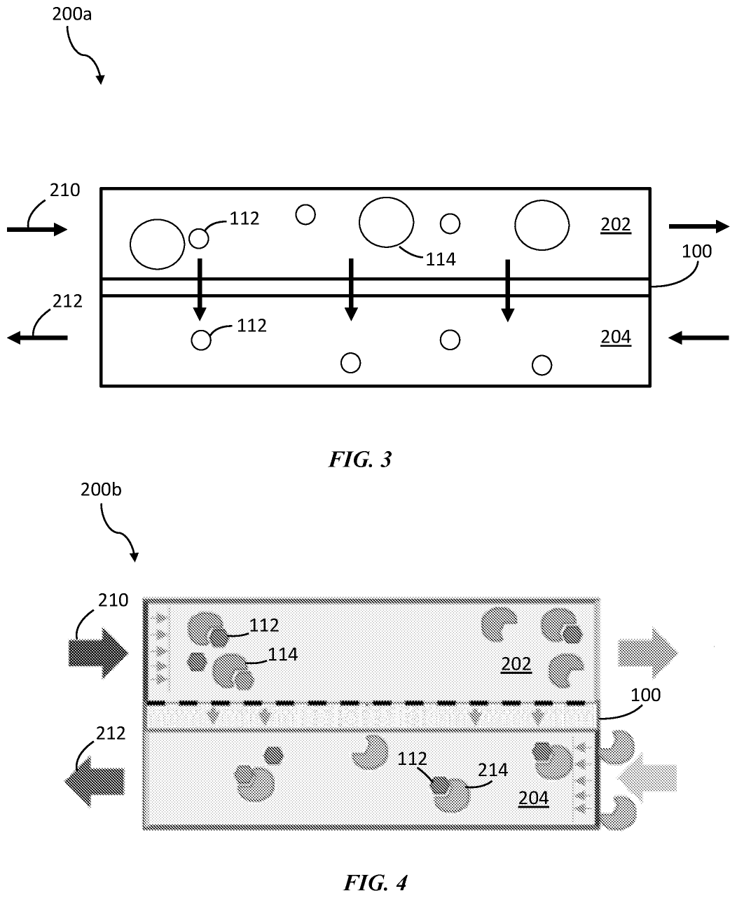

According to certain embodiments, a dialysis system is described herein. FIG. 3 shows, according to some embodiments, a schematic diagram of a dialysis system. Referring to FIG. 3, dialysate system 200a may comprise first compartment 202 configured to receive a flow of blood (e.g., from the body of a patient). In some embodiments, the flow of blood in first compartment 202 is oriented in first direction 210. Dialysate system 200a may comprise second compartment 204 configured to receive a flow of a dialysate (e.g., from a source of dialysate). The flow of the dialysate in second compartment 204 may be oriented in second direction 212 that is substantially opposite first direction 210, which facilitates a diffusive filtration process, though other flow arrangements are also contemplated as the disclosure is not limited in this fashion.

Dialysate system 200a comprises semi-permeable membrane 100 disposed between first compartment 202 and second compartment 204, which functions as explained herein in greater detail. Dialysis system 200a may be configured, for example, such that at least first species 112 is transported from first compartment 202 into second compartment 204 through semi-permeable membrane 100, while restricting at least second species 114 from transporting from first compartment 202 into second compartment 204.

The first species may comprise any of a variety of suitable species, such as, for example, a uremic toxin. Examples of uremic toxins include, but are not limited to, p-cresyl sulfate, indoxyl sulfate, beta-2-microglobulin, urea, creatinine, hippuric acid, 3-carboxy-4-methyl-5-propyl-2-furanpropionic acid, and/or combinations thereof. The second species may comprise any of a variety of suitable species, such as, for example, albumin or immunoglobulins.

Semi-permeable membrane 100 may have any of a variety of configurations, including, but not limited to, concentric tubes, stacked parallel sheets, a spiral configuration, and/or a plate-and-frame configuration. One or more spacers may be disposed between the stacked parallel sheets of semi-permeable membranes, in some embodiments, which may advantageously enhance mixing and mass transfer through the semi-permeable membrane. Spacing between membranes may be, for example, between greater than or equal to 10 micrometers and less than or equal to 10 millimeters. In certain non-limiting embodiments, the porous substrate (e.g., porous substrate 106) may act a spacer between stacked parallel sheets of semi-permeable membranes, while also allowing fluid flow in a direction parallel to the surface of the membrane. The porous substrate may also comprise surface features to enhance mixing while minimizing pressure drop across the support. In some embodiments, the substrate may be constructed such that it has an impermeable layer parallel to the membrane that forms two separate flow pathways (e.g., a first channel formed between the impermeable layer and the intermediate coating, and a second channel formed between the impermeable layer and the atomically thin layer of an adjacent membrane), enabling composite membranes to be stacked one of top of the other without requiring spacer layers in between. In certain embodiments, the substrate is constructed to facilitate separate fluidic connections to the two channels for flowing the blood and dialysate fluids, respectively.

In certain embodiments, the dialysate may comprise an adsorbent that is configured to bind the first species (e.g., a uremic toxin such as p-cresyl sulfate, indoxyl sulfate, and the like). FIG. 4 shows, according to some embodiments, a schematic diagram of dialysis system 200b wherein the dialysate comprises adsorbent 214. In some embodiments, dialysate system 200b comprises second compartment 204 configured to receive a flow of a dialysate-adsorbent mixture, and adsorbent 214 may be configured to adsorb, adhere, bind, and/or complex with first species 112, therefore advantageously improving the removal of first species 112 from first compartment 202 into second compartment 204, and retaining second species 114 (e.g., albumin) in first compartment 202. In some embodiments, the size of adsorbent 214 is larger than the average size of the plurality of pores in the atomically thin active layer to ensure that the adsorbent does not diffuse thorough membrane 100. Although not shown in FIG. 4, adsorbent 214 may be immobilized on the surface of membrane 100 facing second compartment 204. Any of a variety of suitable adsorbents may be employed, including albumin, activated carbon, beta-cyclodextran, and/or functionalized nanoparticles.

According to certain embodiments, a method of performing dialysis is described herein using the membranes and/or dialysis systems. In some embodiments, for example, a method of performing dialysis comprises flowing a first fluid (e.g., blood) in a first compartment across a first surface of a semi-permeable membrane and flowing a second fluid (e.g., a dialysate) in a second compartment across a second surface of the semi-permeable membrane. The first surface may be substantially opposite the second surface, in some embodiments. As described herein, the flow of the first fluid may be in a first direction and the flow of the second fluid may be in a second direction that is substantially opposite the first direction.

According to some embodiments, the method comprises separating at least a first species (e.g., a uremic toxin such as p-cresyl sulfate, indoxyl sulfate, urea, creatinine, and the like) from at least a second species (e.g., albumin) using the semi-permeable membrane. The at least first species may pass through the semi-permeable membrane via selective diffusion, while the at least second species is prevented from passing though the semi-permeable membrane, as described herein.

In certain embodiments, the method further comprises flowing the first fluid out of the first compartment and flowing the second fluid (e.g., comprising the at least first species) out of the second compartment.

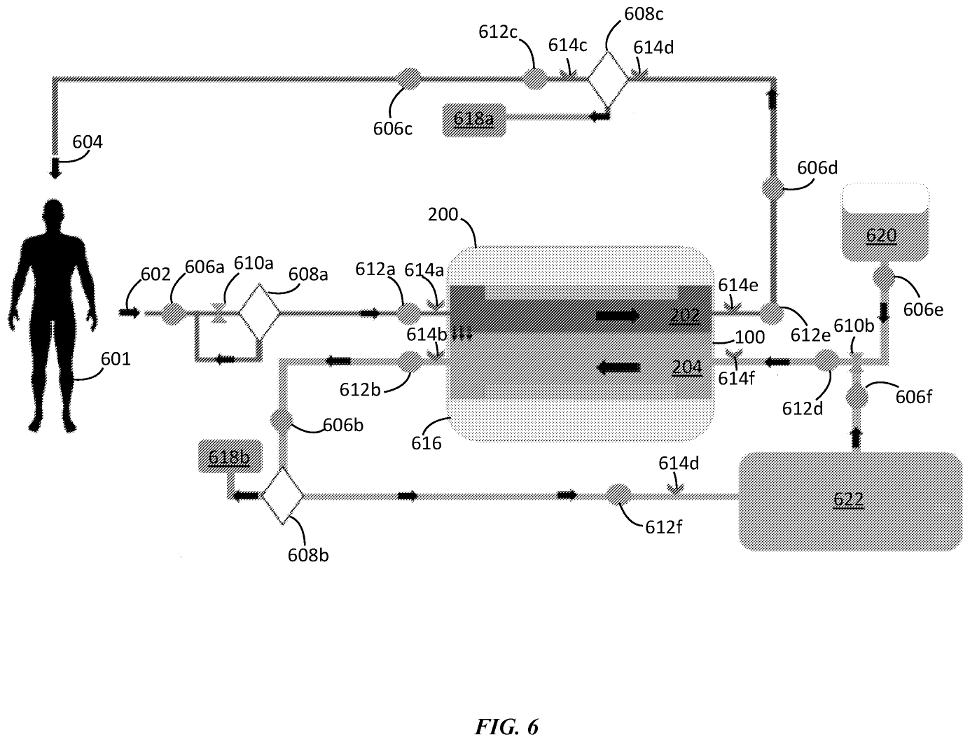

FIG. 6 shows, according to some embodiments, a schematic diagram of a dialysis device. The dialysis device may be configured to operate in any of a variety of suitable modes, including counter-flow, parallel-flow, pressure-driven flow, and/or combinations thereof. Referring to FIG. 6, patient 601 may be fluidically connected to blood inlet 602 (e.g., via needle, syringe, catheter, etc.) to remove blood from patient 601 via pump 606a. Pumps 606 may be used to adjust the flow rate of one or more fluids (e.g., blood and/or the dialysate). The blood may be pre-treated via filter 608a (e.g., with one or more blood-thinners) and flowed to first compartment 202 (e.g., configured to receive the flow of blood) of dialysis system 200. Valve 610a may be positioned along the flow path from blood inlet 602 to filter 608a, and pressure gauge 612a and sampling port 614a may be positioned along the blood flow path from filter 608a to first compartment 202 of dialysis system 200. Pressure gauges 612 may be used to monitor the pressure of the system, and sampling ports 614 may be used for open-loop or closed-loop feedback control.

Clean dialysate from dialysate source 620 may flow to second compartment 204 of dialysate system 200 via pump 606e. Adsorbent source 622 may be coupled to the flow of the clean dialysate via pump 606f and valve 610b to provide a source of adsorbents that may be used to enhance the removal of a target species from blood, as explained herein, resulting in a dialysate-adsorbent mixture that is flowed to second compartment 204 of dialysis system 200. Pressure gauge 612d and/or sampling port 614f may be positioned along the dialysate and/or dialysate-adsorbent mixture flow path from dialysate source 620 and/or adsorbent source 622 to dialysis system 200.

Dialysis system 200 functions as described herein, for example, to separate at least a first species from at least a second species by transporting the first species from first compartment 202 into second compartment 204 through membrane 100 (e.g., by diffusion). Dialysis system 200 may comprise thermostasis bath 616 configured to maintain and/or regulate the temperature of dialysis system 200. Although not shown in the figures, dialysis system 200 may comprise one or more viewports to allow analytical measurements such as spectroscopy and/or imaging.

The contaminated dialysate flows out of second compartment 204 of dialysate system 200 via pump 606b and flows to filter 608b, which is configured to recover the dialysate and/or the adsorbents. The adsorbents may be recovered, in some embodiments, by changing the pH, pressure, and/or temperature of the dialysate, by applying an electric potential to induce a change in the interaction between the adsorbent and the species removed from the blood, and/or by a separate dialysis technique. The recovered adsorbent may be subsequently flowed to adsorbent source 622, and the recovered dialysate may be flowed to waste recovery 618b. In an alternate embodiment, the recovered dialysate may be returned to dialysate source 620. In the case of a membrane immobilized adsorbent, as explained above, the adsorbent may be regenerated by introducing a toxin free solution after the dialysis to remove target species bound to the adsorbent, or by changing the binding affinity between the adsorbent and the bound target species, by, for example, electrochemical-mediated redox. Sampling port 614b and pressure gauge 612b may be positioned along the flow path from dialysis system 200 to filter 608b, and pressure gauge 612f and sampling port 614d may be positioned along the flow path from filter 608b to adsorbent source 622.

The blood that has been subjected to dialysis may flow out of first compartment 202 via pump 606d to filter 608c, which is configured to treat the blood before returning to patient 601 (e.g., to remove one or more blood-thinners, which may flow to waste recovery 618a). Sampling ports 614e and 614d and pressure gauge 612e may be positioned along the flow path from dialysis system 200 to filter 608c. The filtered blood may then flow to blood outlet 604 fluidically connected to patient 601 via pump 606c. Sampling port 614c and pressure gauge 614c may be positioned along the flow path from filter 608c to blood outlet 604.

The dialysis scheme shown in FIG. 6 may have a variety of alternate configurations, as the disclosure is not meant to be limited to the embodiment shown in FIG. 6. In certain embodiments, for example, multiple semi-permeable membranes may be employed between first compartment 202 and second compartment 204 (e.g., a stack of two or more semi-permeable membranes). Multiple dialysis systems 200 may be employed in series, in certain embodiments.

In some embodiments, a source of protein-bound uremic toxins may be employed in the dialysis system and used for modeling a patient's generation rate of toxins, but not used during patient care.

The following examples are intended to illustrate certain embodiments of the present disclosure, but do not exemplify the full scope of the disclosure.

### Example 1

The following example describes how the fabrication of a composite membrane used in a dialysis device.

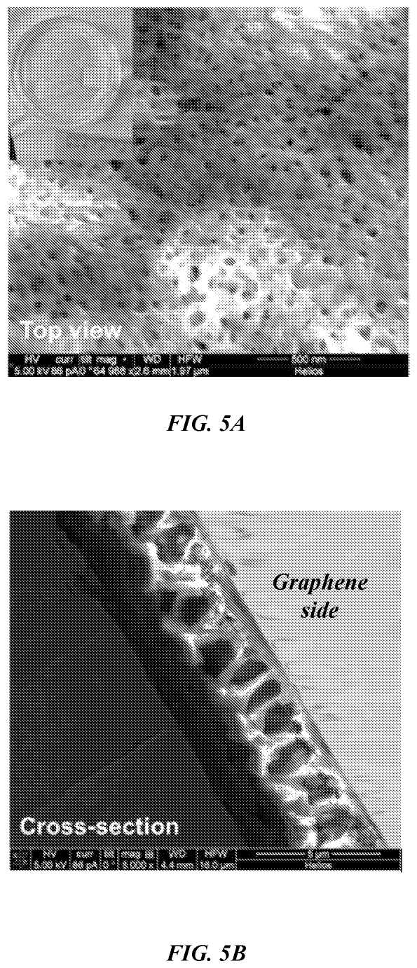

Chemically vapor deposited (CVD) graphene on copper (G-Cu) were purchased from Graphenea. A porous coating was formed on the atomically thin graphene layer using a casting solution having the following composition: polyethersulfone (15 wt. %), N-methyl pyrrolidone (83 wt. %, Sigma-Aldrich Reagent Plus, 99%) and methanol (2 wt. %, Macron, anhydrous). The casting solution was spin-coated on the atomically thin graphene layer supported on copper at 4000 rpm for 10 seconds under ambient conditions. In using the pipet to drop the PES solution onto the graphene surface, care was taken to ensure the PES covered the entire surface of the G-Cu but did not spill over. The PES-G-Cu piece was immediately placed into a 50:50% water:isopropanol bath for phase inversion to form the polymer layer for at least 2 hours. After phase inversion in the anti-solvent bath of a water/isopropanol mixture with a volumetric ratio of 1:1, a thin coating was formed on the graphene surface that has a pore size range of 50-200 nm (see FIGS. 5A and 5B). Selective pores on graphene were generated via oxygen plasma at 500 mTorr over various durations using a plasma cleaner (Harrick Plasma PDC-001, maximum power 30 W).

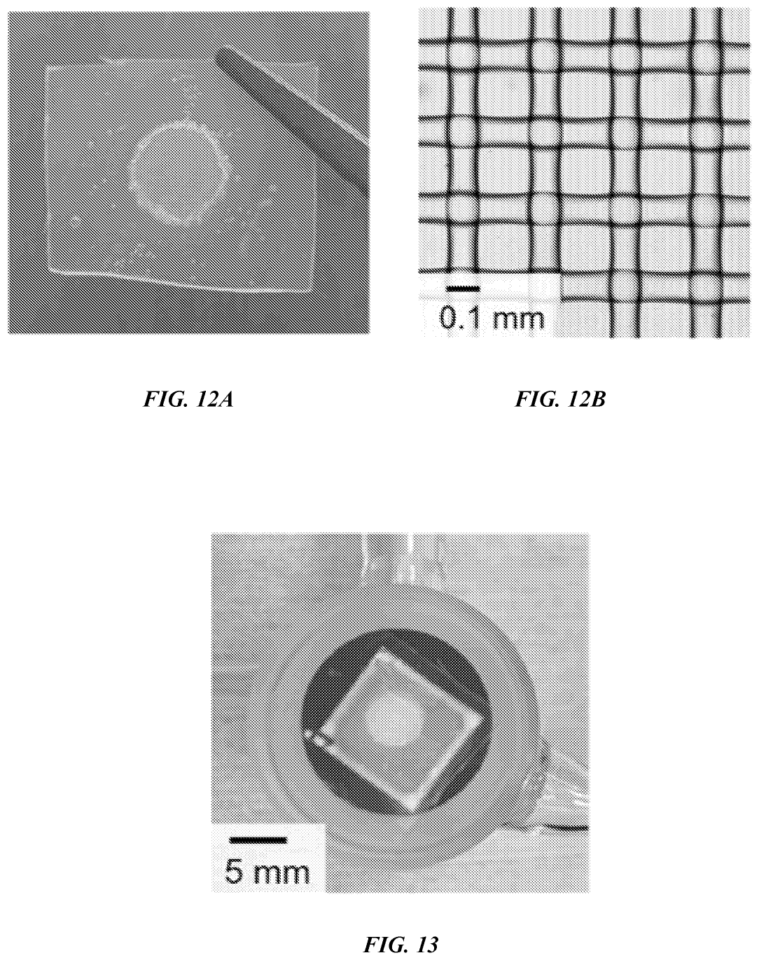

A 127×127 nylon mesh (McMaster Carr) was used as a support. Because of its woven structure, edges around the active support area were sealed to avoid leakage. Polydimethylsiloxane (PDMS; Dow Sylgard 184 Silicone Elastomer) was used as the sealant. To create the orifice, circular pieces of Kapton tape with 5 mm diameter (size of the diffusion cell orifice) were punched out and pressed onto the mesh. A thin film of PDMS was then cast on the mesh, barely covering the tape. The tape prevented PDMS from accessing the circular active area. The PDMS were then heated to 70° C. in an oven and cured for 2 hours, and the tape was removed. The nylon mesh substrate with its edges sealed by polydimethylsiloxane (see FIGS. 12A and 12B) was then attached to the polyethersulfone side of the membrane, forming the composite membrane (see FIG. 13).

Depending on the stickiness of the support, the support could be (1) directly attached onto the copper before etching if the support was sticky or (2) attached after the copper was etched away. For (1): The support was stamped directly onto the PES-G-Cu. To improve adhesion, a thin layer of glue (e.g., epoxy) could be added to the nylon mesh by gently pressing the nylon mesh onto a thinly casted layer of glue on a glass slide before the stamping process. However, the thickness of the glue was hard to control even with spin-coating and if it was too thick it could block pores in the support. The support-PES-G-Cu composite was placed, with Cu side facing down, into an APS-100 copper etchant (Transene) solution for 5 minutes to remove potential graphene residue on the backside of the copper. The composite was then transferred into a 1:5 water-diluted APS-100 solution to continue the etching overnight at a lower rate to prevent substantial bubble formation which could damage the graphene surface. The remaining support-PES-G composite was then washed thrice in large water baths and air dried. For (2): The PES-G-Cu composite was placed into the APS-100 solution with Cu side facing down for 5 minutes, and transferred into the 1:5 water-diluted APS-100 to etch away the Cu overnight. The resulting PES-G would have the graphene facing the solution. The support was placed on top of the PES. Because the support was not sticky, the support-PES-G had to be pushed beneath the water, flipped 180° such that graphene was facing the top, and scooped out of the bath gently and dried. Excessive agitation or perturbation to the membrane should be avoided. The latter process is less desirable as it could introduce defects to the graphene surface due to the mechanical stresses imparted on the composite during fabrication. The polyethersulfone was then placed onto the nylon mesh. The polydimethylsiloxane used to seal the edges of the nylon mesh also aids with support adhesion with the polyethersulfone.

The configuration of the final composite membrane (see FIG. 13) includes a nanoporous graphene membrane, with pores generated using oxygen plasma, on top of a polyethersulfone intermediary layer formed through interfacial polymerization, on top of a nylon mesh orifice sealed by polydimethylsiloxane at the edge. The polyethersulfone shows the desired hierarchical pore structure with smaller pores around 20-100 nm on the surface for better adhesion with graphene and microbe rejection, and larger pores within the membrane to facilitate transport, with a thickness of ˜2 μm.

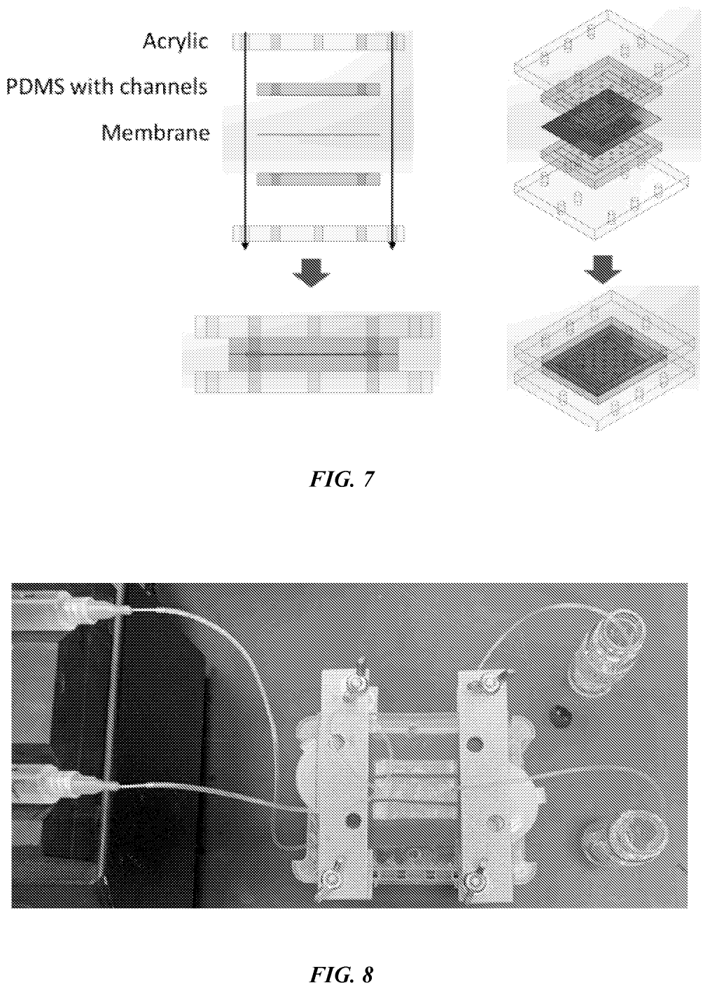

A dialysis device prototype with channel thickness of 125 micrometers was fabricated by casting polydimethylsiloxane onto a mold made using scotch tape, with holes punched at both ends to enable fluid entrance and exit. To create the device, the mold was created by placing various layers scotch tape (62.5 μm thick) on top of a large petri dish and cutting the scotch tape into the desired negative, following a method that allows for simple fabrication of microfluidic devices. The main channels had heights of 125 μm (2×tape) and widths and lengths of 3 cm. 2 mm diameter pillars that are 125 μm tall were used to support the membrane and prevent collapse of the channels. The inlet and outlet sections contained a 625 μm tall (10×tape) reservoir such that the feed and permeate solution can spread across the width of the channels. PDMS was then casted onto the mold and cured at 85° C. overnight, before being peeled off for assembly, where 0.635 cm thick acrylic plates and silicone gaskets to even out the pressure distribution were used to press the PDMS channels and membrane together to form the flow device. The composite membrane was then placed in between two of such polydimethylsiloxane channels, and transparent acrylic plates were pressed onto the structure to seal the channels while enabling observation of fluid flow. Tubes were connected to the entrances and exits of the channels and a syringe pump was used to pump fluid through the device. See, for example, the computer-aided design (CAD) of the dialysis device shown in FIG. 7 and the dialysis device shown in FIG. 8.

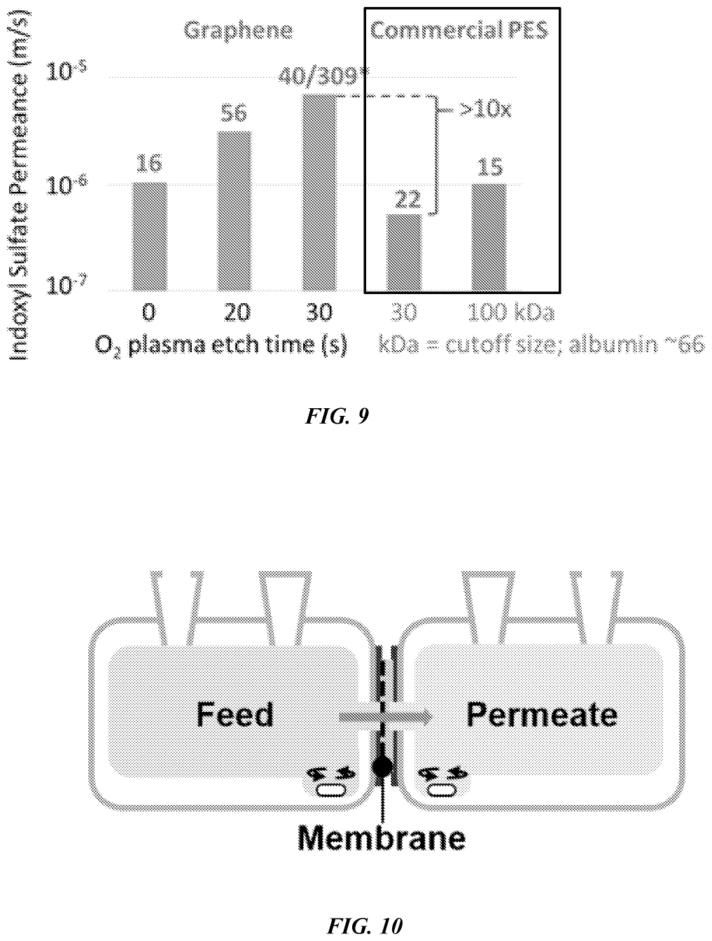

Protein-bound uremic toxin transport was measured through the membrane. The membrane was mounted in a stirred diffusion cell with one side filled with phosphate buffered saline (PBS) and the other with either indoxyl sulfate (IS) in PBS or bovine serum albumin (BSA) in PBS. The concentration of the solute was measured to extract the permeance, defined as the mass transport per unit membrane area per unit time, divided by concentration difference across the membrane. The permeance to IS and selectivity between IS and BSA increased with oxygen plasma treatment time, indicating formation of selective pores. The fabricated membranes had a greater permeance than commercially-obtained PES membranes, measured in the same way and shown as reference in FIG. 9.

### Example 2

The following example describes the formation of an intermediate coating by phase inversion of a spin-coated polymer film.

In a phase inversion process, a skin layer develops on top of a cast film as a result of rapid solvent loss because the top layer contacts the coagulation bath ahead of the sublayers. The skin layer typically has a different porous structure compared to the sublayers because its formation influences diffusion of solvent and nonsolvent in the sublayers, altering the kinetics of demixing and thereby the porous structures developed in the sublayers. Often, the skin layer is dense, with very small pores or even no pores. For semi-permeable membranes, an intermediate coating with a dense skin layer formed on the opposite side of the atomically thin selective layer would compromise both permeance and selectivity of the membrane. Several parameters, however, have proved effective in mitigating the formation of such a distinguishable skin layer.

Use of a highly miscible solvent/non-solvent combination (e.g., n-methyl-2-pyrrolidone (NMP) and water) yields a skin layer with large and connected pores in the layer. In this example, the composition (polymer, solvent, and nonsolvent) profile in the top layer touches or crosses the binodal line in the ternary phase diagram quickly due to rapid exchange of solvent and nonsolvent; instantaneous de-mixing happens which means the polymer precipitates and a skin layer with a highly porous structure is formed very rapidly; and this occurs generally with highly miscible solvent/non-solvent systems. A skin layer with a highly porous structure offers less hindrance to the subsequent diffusion of solvent and non-solvent in the sublayers. As a result, de-mixing in the sublayers follows the similar kinetics as the top layer, engendering a similar porous structure.

Matching the viscosity of solvent and non-solvent balances the rates of their exchange in the cast film, suppressing the formation of large “finger-like” pores or microvoids in the sublayers. In the case where the viscosity of solvent is significantly higher than that of the nonsolvent, the phase separation is dominated by influx of the nonsolvent, giving rise to a large volume fraction of the polymer-poor phase, which will later develop into pores in the sublayers. By adding additives, such as isopropanol alcohol (viscosity: 2.05 cP at 25° C.) into water (viscosity 0.89 cP at 25° C.) up to an equal volume ratio, the mixture viscosity is increased and approaches that of NMP (1.89 cP at 25° C.). In addition, isopropanol alcohol is a much less effective non-solvent for polyethersulfone (PES) than water, because the polymer viscosity does not increase as much in isopropanol compared to that in water. Therefore, an anti-solvent with such a composition slows down polymer precipitation in the top layer, allowing more time for the porous structure to develop and equilibrate with the sublayers while balancing the exchange of solvent and non-solvent. This yields a small “sponge-like” and uniformly porous film, in contrast to a skin layer with much smaller pores on the top surface.

Adding non-solvents as additives to the polymer casting solution seeds nuclei for developing small and uniform pores. In some embodiments, a trace amount (e.g., 2 wt. %) of methanol is added to the casting solution (e.g., 15 wt. % PES, 83 wt. % NMP). This composition does not touch the binodal line and all components exist as a single uniform phase. The non-solvent, methanol, is dissolved in the casting solution. The methanol provides nucleation sites for pore formation during the subsequent phase inversion, providing a more finely distributed, uniform porous film.

A distinguishable skin layer is the result of a different phase separation and precipitation between the top layer and sublayers. The thermodynamics of phase separation is characterized and well-illustrated by the phase diagram; but it is strongly influenced by the kinetics which varies along the thickness of the cast film. As such, the intermediate coating of the semi-permeable membrane described herein may have, in some embodiments, a thickness in a range of 1 to 8 micrometers, which is much thinner than the thickness of conventional films formed by phase inversion (e.g., 60 to 80 micrometers). A thin intermediate coating has more uniform pores as compared to phase inversion of a thick polymer layer, since the solvent exchange even on the bottom surface is relatively rapid compared to the case where a thick layer undergoes phase inversion by solvent exchange.

In addition to phase inversion by solvent exchange, other methods such as phase inversion by temperature change may also be used to produce the intermediate layer.

### Example 3

The following example describes the diffusion performance of the composite membrane fabricated in Example 1.

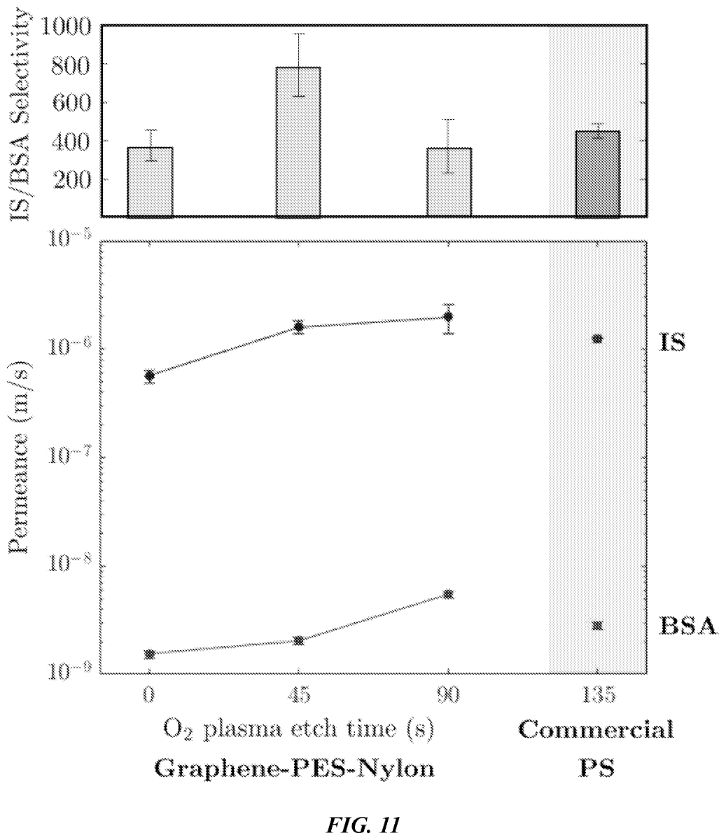

Single-component diffusion tests with indoxyl sulfate (IS) and bovine serum albumin (BSA) were performed to characterize the separation performance of the 3-layer composite membrane (Nylon-PES-G) with various chemical etch durations. FIG. 10 shows the diffusion test set-up and FIG. 11 shows the permeance and selectivity performance of the composite membrane compared against polysulfone (PS) hollow fiber membranes taken from a commercial dialyzer. The dialysis membrane should have high toxin permeance such that the toxin could be removed from the feed (plasma), while having low albumin permeance such that there is minimal protein loss during dialysis.

As the oxygen plasma etch time increases, both IS and BSA permeances increase. The increase is more rapid for IS compared to BSA due to IS's smaller size, and thus IS/BSA selectivity increases initially at short etch times. This is the regime where the nanopores in graphene dominates the diffusive transport resistance. However, at longer etch times, IS permeance increases less and seems to plateau, suggesting that the support begins to dominate the IS resistance, whereas BSA permeance continues to rise. This results in a drop in selectivity. Thus, the highest selectivity is achieved at an intermediate etch time of ˜45 seconds.

The highest selectivity achieved by the 3-layer nanoporous graphene composite membrane at 45 seconds etch was slightly less than twice that that of a commercial PS membrane. The IS permeance was also slightly higher. The expected albumin loss over a 4 hour dialysis session for a typical membrane with area 1.8 m² and a plasma albumin concentration of 4 g/dL would be 2.1 g for the 3-layer composite membrane and 2.9 g for the commercial PS membrane, both of which are lower than the recommended limit of 4 g albumin loss per treatment (although clinical trials show no hard evidence of physiological damage if the loss is <20 g). The results show great promise for the 3-layer structure membrane to be utilized for hemodialysis to improve the removal of protein bound uremic toxins.

The embodiments described herein may be embodied as a method, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Further, some actions are described as taken by a “user.” It should be appreciated that a “user” need not be a single individual, and that in some embodiments, actions attributable to a “user” may be performed by a team of individuals and/or an individual in combination with computer-assisted tools or other mechanisms.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present disclosure.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A semi-permeable membrane, the semi-permeable membrane comprising:
an atomically thin layer, wherein the atomically thin layer comprises a plurality of pores that allow transport of at least a first species though the semi-permeable membrane while restricting transport of at least a second species through the semi-permeable membrane;
a porous intermediate coating disposed on the atomically thin layer; and
a porous substrate, wherein the porous intermediate coating is disposed between the atomically thin layer and the porous substrate.

2. The semi-permeable membrane of claim 1, wherein the atomically thin layer comprises graphene, boron nitride, and/or molybdenum disulfide.

3. The semi-permeable membrane of claim 1, wherein the atomically thin layer has an average thickness between greater than or equal to 0.1 nm and less than or equal to 1 nm.

4. The semi-permeable membrane of claim 1, wherein the plurality of pores of the atomically thin layer have an average pore size between greater than or equal to 1 nm and less than or equal to 3.8 nm.

5. The semi-permeable membrane of claim 1, wherein the porous intermediate coating comprises polyethersulfone and/or polysulfone.

6. The semi-permeable membrane of claim 1, wherein the porous intermediate coating has an average thickness between greater than or equal to 1 micrometer and less than or equal to 3 micrometers.

7. The semi-permeable membrane of claim 1, wherein the porous intermediate coating comprises a plurality of pores having an average pore size between greater than or equal to 20 nm and less than or equal to 200 nm.

8. The semi-permeable membrane of claim 1, wherein the porous substrate comprises nylon and/or silicon.

9. The semi-permeable membrane of claim 1, wherein the porous substrate has an average thickness between greater than or equal to 50 micrometers and less than or equal to 150 micrometers.

10. The semi-permeable membrane of claim 1, wherein the porous substrate comprises a plurality of pores having an average pore size between greater than or equal to 100 micrometers and less than or equal to 500 micrometers.

11. A dialysis system, the dialysis system comprising:
a first compartment configured to receive a flow of blood;
a second compartment configured to receive a flow of a dialysate; and
a semi-permeable membrane disposed between the first compartment and the second compartment, the semi-permeable membrane comprising:
an atomically thin layer, wherein the atomically thin layer comprises a plurality of pores that allow transport of at least a first species though the semi-permeable membrane while restricting transport of at least a second species through the semi-permeable membrane;
a porous intermediate coating disposed on the atomically thin layer; and
a porous substrate, wherein the porous intermediate coating is disposed between the atomically thing layer and the porous substrate,
wherein the dialysis system is configured such that at least the first species is transported from the first compartment into the second compartment though the semi-permeable membrane.

12. The dialysis system of claim 11, wherein the flow of blood in the first compartment is oriented in a first direction and the flow of the dialysate in the second compartment is oriented in a second direction that is substantially opposite the first direction.

13. The dialysis system of claim 11, wherein the dialysate comprises an adsorbent configured to bind at least the first species.

14. The dialysis system of claim 11, wherein the atomically thin layer has an average thickness between greater than or equal to 0.1 nm and less than or equal to 1 nm.

15. The dialysis system of claim 11, wherein the plurality of pores of the atomically thin layer have an average pore size between greater than or equal to 1 nm and less than or equal to 3.8 nm.

16. The dialysis system of claim 11, wherein the porous intermediate coating has an average thickness between greater than or equal to 1 micrometer and less than or equal to 3 micrometers.

17. The dialysis system of claim 11, wherein the porous intermediate coating comprises a plurality of pores having an average pore size between greater than or equal to 20 nm and less than or equal to 200 nm.

18. A method of performing dialysis, the method comprising:
separating at least a first species from at least a second species using a semi-permeable membrane, the semi-permeable membrane comprising:
an atomically thin layer, wherein the atomically thin layer comprises a plurality of pores that allow transport of at least the first species though the semi-permeable membrane while restricting transport of at least the second species through the semi-permeable membrane;
a porous intermediate coating disposed on the atomically thin layer; and
a porous substrate, wherein the porous intermediate coating is disposed between the atomically thin layer and the porous substrate,
wherein at least the first species passes through the semi-permeable membrane via diffusion.

19. The method of claim 18, wherein the first species comprises a uremic toxin and the second species comprises albumin.

20. The method of claim 18, wherein the atomically thin layer has an average thickness between greater than or equal to 0.1 nm and less than or equal to 1 nm.

21. The method of claim 18, wherein the plurality of pores of the atomically thin layer have an average pore size between greater than or equal to 1 nm and less than or equal to 3.8 nm.

22. The method of claim 18, wherein the porous intermediate coating has an average thickness between greater than or equal to 1 micrometer and less than or equal to 3 micrometers.

23. The method of claim 18, wherein the porous intermediate coating comprises a plurality of pores having an average pore size between greater than or equal to 20 nm and less than or equal to 200 nm.
